(19) United States
(12) Patent Application Publication
(10) Pub. No.: US 2009/0054783 A1
(43) Pub. Date: Feb. 26, 2009

(54) ULTRASOUND DIAGNOSTIC APPARATUS, ULTRASOUND PROBE, AND TEMPERATURE CALCULATING METHOD

(76) Inventor: Hiroaki Shibata, Tokyo (JP)

Correspondence Address: PATRICK W. RASCHE (20459);ARMSTRONG TEASDALE LLP, ONE METROPOLITAN SQUARE, SUITE 2600, ST. LOUIS, MO, 63102-2740, US

(21) Appl. No.: 12/196,980

(22) Filed: Aug. 22, 2008

(30) Foreign Application Priority Data
Aug. 24, 2007 (JP) 2007-217693

(51) Int. Cl. A61B 8/00

(52) U.S. Cl. 600/459

(57) ABSTRACT

An ultrasound diagnostic apparatus includes a temperature sensor disposed on an ultrasound probe to detect the temperature of the surface of the probe, a calculating unit which calculates the temperature on the basis of an input from the temperature sensor, and a storage unit in which the temperature characteristics of said temperature sensor measured in advance are stored. Said calculating unit performs temperature calculation according to the temperature characteristics of said temperature sensor stored in said storage unit.

### CROSS REFERENCE TO RELATED APPLICATIONS

This application claims the benefit of Japanese Patent Application No. 2007-217693 filed Aug. 24, 2007, which is hereby incorporated by reference in its entirety.

### BACKGROUND OF THE INVENTION

The subject matter disclosed herein relates to an ultrasound diagnostic apparatus, an ultrasound probe, a method of calculating the temperature of the surface of an ultrasound probe in an ultrasound diagnostic apparatus, and a method of processing echo reception signals in an ultrasound diagnostic apparatus.

An ultrasound diagnostic apparatus is provided with an ultrasound probe having transducers formed of a piezoelectric material, and is so composed that an ultrasound wave is transmitted to a subject by applying a voltage to the transducers and echoes responding thereto are received by the transducers to generate an image of the inside of the subject.

In such an ultrasound diagnostic apparatus, the more the driving power of the transducers is increased, the more the calorific value attributable to the internal loss in the ultrasound probe increases, and the surface temperature of the ultrasound probe in contact with the subject rises.

Therefore, there is a practice to dispose on the ultrasound probe a temperature sensor for detecting the surface temperature, and to so control the driving power of the transducers on the basis of the detection value of this temperature sensor as not to allow the surface temperature of the ultrasound probe to surpass a prescribed temperature (see Japanese Unexamined Patent Publication No. Hei 8(1996)-56942 for instance).

Incidentally, in temperature detection by the temperature sensor, where a thermistor is used as temperature sensor, the temperature is detected on the basis of its electrical resistance. More specifically, the electrical resistance of the thermistor is measured, and the temperature is calculated by substituting this electrical resistance into the relationship between the temperature and the electrical resistance in the thermistor.

The relationship between the temperature and the electrical resistance often varies from one thermistor to another. However, as a fixed function is used as the primary function for calculating the temperature, namely the temperature calculation function, the calculated temperature may differ from the actual temperature. Since in such a case the transmission power of the ultrasound wave pulse is controlled on the basis of a wrong detection value, the surface temperature of the ultrasound probe may surpass the prescribed temperature.

Further, various frequency bands are set for the ultrasound probe according to the purpose of use or the like, ultrasound probes are so manufactured as to have a set frequency characteristic. However, the frequency characteristics of individual transducers are not exactly the same, but differ within a prescribed extent. If there are variations in frequency characteristic among individual transducers, there are likely to be more fluctuations in brightness and resolution in the images obtained than where there are no fluctuations, resulting in a fear of deterioration of image quality.

Furthermore, since the ultrasound transmission power or the like differs on account of individual variances of the transducers, the time response characteristics of individual transducers (the relationship between the length of time from the transmission and reception of the ultrasound wave and the sensitivity) are also uneven. Where the time response characteristics of individual transducers are uneven, there also are likely to be fluctuations in brightness and resolution in the images obtained, resulting in a fear of deterioration of image quality.

### BRIEF DESCRIPTION OF THE INVENTION

It is desirable that the problems described previously are solved.

According to the embodiments described herein, accurate determination of the surface temperature of the ultrasound probe is allowed. Also, image quality is improved.

The invention in its first aspect provides an ultrasound diagnostic apparatus including: a temperature sensor disposed on an ultrasound probe to detect the temperature of the surface of the probe; and a calculating unit which calculates the temperature on the basis of an input from the temperature sensor, the ultrasound diagnostic apparatus further including a storage unit in which the temperature characteristics of the temperature sensor measured in advance are stored, wherein the calculating unit performs temperature calculation according to the temperature characteristics of the temperature sensor stored in the storage unit.

The invention in its second aspect provides an ultrasound diagnostic apparatus according to the first aspect, in which the calculating unit performs temperature calculation by substituting a physical quantity, which varies according to the temperature at the temperature sensor, into a temperature calculation function representing the relation between the physical quantity and the temperature of the temperature sensor, wherein the storage unit stores a physical quantity at a specific temperature of the temperature sensor measured in advance, the ultrasound diagnostic apparatus being further provided with a deriving unit which derives the temperature calculation function on the basis of a physical quantity stored in the storage unit.

The invention in its third aspect provides an ultrasound diagnostic apparatus according to the second aspect described above, wherein the physical quantity of the temperature sensor which varies according to the temperature is an electrical resistance, electrical resistances at two or more different specific temperatures measured in advance being stored in the storage unit; the deriving unit derives a temperature calculation function representing the relationship between electrical resistances and temperatures at the temperature sensor on the basis of the electrical resistances at the two or more different specific temperatures stored in the storage unit; and the calculating unit performs temperature calculation by substituting electrical resistances at the temperature sensor into the temperature calculation function.

The invention in its fourth aspect provides an ultrasound diagnostic apparatus according to the first, second and third aspect described above, wherein the storage unit is provided in the ultrasound probe.

The invention in its fifth aspect provides an ultrasound probe provided with a temperature sensor for detecting the temperature of the surface of the probe, further including a storage unit in which temperature characteristics of the temperature sensor measured in advance are stored to perform temperature calculation matching the temperature characteristics of the temperature sensor.

The invention in its sixth aspect provides a temperature calculating method for use in an ultrasound diagnostic apparatus which is provided with a temperature sensor disposed on an ultrasound probe to detect the temperature of the surface of the probe and calculates the temperature on the basis of an input from this temperature sensor, wherein temperature calculation matching the temperature characteristics of the temperature sensor measured in advance is performed.

The invention in its seventh aspect provides a temperature calculating method for use in an ultrasound diagnostic apparatus according to the sixth aspect described above, wherein temperature calculation is performed by substituting a physical quantity, which varies according to the temperature at the temperature sensor, into a temperature calculation function representing the relation between the physical quantity and the temperature of the temperature sensor, and the temperature calculation function is derived by using a physical quantity at a specific temperature of the temperature calculation function measured in advance.

The invention in its eighth aspect provides a temperature calculating method for use in an ultrasound diagnostic apparatus according to the seventh aspect described above, wherein the temperature calculation function is derived by using electrical resistances at two or more different specific temperatures being measured in advance; and temperature calculation is performed by substituting an electrical resistance at the temperature sensor into the temperature calculation function.

The invention in its ninth aspect provides an ultrasound diagnostic apparatus including an ultrasound probe provided with a plurality of transducers which transmit and receive ultrasound waves; and a receiver unit which receives as echo reception signals a reflected wave received by the transducers, further provided with a storage unit in which the frequency characteristic of each of the transducers measured in advance is stored; and a correcting unit which corrects the echo reception signal of each of the transducers on the basis of the frequency characteristic of each of the transducers stored in the storage unit.

The invention in its tenth aspect provides an ultrasound diagnostic apparatus according to the ninth aspect described above, wherein the correction of echo reception signals by the correcting unit is accomplished on the basis of comparison of the frequency characteristic stored in the storage unit and the set frequency characteristic of the ultrasound probe.

The invention in its eleventh aspect provides an ultrasound diagnostic apparatus according to the ninth or tenth aspect described above, wherein the storage unit in which the frequency characteristic of each of the transducers measured in advance is stored is disposed in the ultrasound probe.

The invention in its twelfth aspect provides an ultrasound probe provided with a plurality of transducers which transmit and receive ultrasound waves, further provided with a storage unit in which the frequency characteristic of each of the transducers measured in advance is stored to perform correction matching the frequency characteristics of the transducers regarding the echo reception signals received by the transducers.

The invention in its thirteenth aspect provides a signal processing method for echo reception signals for use in an ultrasound diagnostic apparatus including an ultrasound probe provided with a plurality of transducers which transmit and receive ultrasound waves; and a receiver unit which receives as echo reception signals a reflected wave received by the transducers, wherein the echo reception signal of each of the transducers is corrected on the basis of the frequency characteristic of each of the transducers measured in advance.

The invention in its fourteenth aspect provides a signal processing method for use in an ultrasound diagnostic apparatus according to the thirteenth aspect described above, wherein correction of the echo reception signals is accomplished on the basis of comparison of the frequency characteristic of each of the transducers measured in advance and the set frequency characteristic of the ultrasound probe.

The invention in its fifteenth aspect provides an ultrasound diagnostic apparatus including an ultrasound probe provided with a plurality of transducers which transmit and receive ultrasound waves; and a receiver unit which receives as echo reception signals a reflected wave received by the transducers, the ultrasound diagnostic apparatus further including a storage unit in which the time response characteristic of each of the transducers measured in advance is stored; and a correcting unit which corrects the echo reception signal of each of the transducers on the basis of the time response characteristic of each of the transducers stored in the storage unit.

The invention in its sixteenth aspect provides an ultrasound diagnostic apparatus according to the fifteenth aspect described above, wherein the correction of echo reception signals by the correcting unit is accomplished on the basis of comparison of the time response characteristic stored in the storage unit and the standard time response characteristic of the ultrasound probe.

The invention in its seventeenth aspect provides an ultrasound diagnostic apparatus according to the fifteenth or sixteenth aspect, wherein the storage unit in which the time response characteristic of each of the transducers measured in advance is stored is disposed in the ultrasound probe.

The invention in its eighteenth aspect provides an ultrasound probe including a plurality of transducers which transmit and receive ultrasound waves, the ultrasound probe further provided with a storage unit in which the time response characteristic of each of the transducers measured in advance is stored to perform correction matching the time response characteristics of the transducers regarding the echo reception signals received by the transducers.

The invention in its nineteenth aspect provides a signal processing method for echo reception signals for use in an ultrasound diagnostic apparatus including: an ultrasound probe provided with a plurality of transducers which transmit and receive ultrasound waves; and a receiver unit which receives as echo reception signals a reflected wave received by the transducers, wherein each of echo reception signals is corrected on the basis of the time response characteristic of each of the transducers measured in advance.

The invention in its twentieth aspect provides a signal processing method for use in an ultrasound diagnostic apparatus according to the nineteenth aspect, wherein correction of the echo reception signals is accomplished on the basis of comparison of the time response characteristic of each of the transducers measured in advance and the standard time response characteristic of the ultrasound probe.

According to the first and sixth aspects of the invention, since temperature calculation is performed according to the temperature characteristics of the temperature sensor measured in advance, the surface temperature of the ultrasound probe can be accurately determined.

According to the second and seventh aspects of the invention, since the temperature calculation function is derived on the basis of a physical quantity at a specific temperature measured in advance with respect to the temperature sensor, an accurate temperature calculation function can be derived with respect to the temperature sensor. Since temperature calculation according to the temperature characteristics of the temperature sensor can be performed by calculating the temperature by using such a temperature calculation function, the surface temperature of the ultrasound probe can be accurately determined.

According to the third and eighth aspects of the invention, since the temperature calculation function is derived on the basis of electrical resistances at two or more different specific temperatures measured in advance with respect to the temperature sensor, an accurate temperature calculation function can be derived with respect to the temperature sensor. Since temperature calculation according to the temperature characteristics of the temperature sensor can be performed by calculating the temperature by using such a temperature calculation function, the surface temperature of the ultrasound probe can be accurately determined.

According to the fourth aspect of the invention, temperature calculation matching the temperature characteristics of the temperature sensor stored in the storage unit disposed on the ultrasound probe can be performed.

According to the fifth aspect of the invention, since temperature calculation matching the temperature characteristics of the temperature sensor measured in advance can be performed, the surface temperature of the ultrasound probe can be accurately determined.

According to the ninth and thirteenth aspects of the invention, since the echo reception signal of each of the transducers is corrected on the basis of the frequency characteristic of each of the transducers measured in advance, even if the frequency characteristics are uneven among the transducers, image quality can be enhanced.

According to the tenth and fourteenth aspects of the invention, since the echo reception signal of each of the transducers is corrected on the basis of comparison of the frequency characteristic stored in the storage unit and the set frequency characteristic of the ultrasound probe, even if the frequency characteristics are uneven among the transducers, image quality can be enhanced.

According to the eleventh aspect of the invention, correction of the echo reception signals matching the frequency characteristics of the transducers stored in the storage unit disposed on the ultrasound probe can be performed.

According to the twelfth aspect of the invention, since correction of the echo reception signals matching the frequency characteristics of the transducers measured in advance is made possible, image quality can be enhanced.

According to the fifteenth and nineteenth aspects of the invention, since the echo reception signal of each of the transducers is corrected on the basis of the time response characteristic of each of the transducers measured in advance, even if the time response characteristics are uneven among the transducers, image quality can be enhanced.

According to the sixteenth and twentieth aspects of the invention, since the echo reception signal of each of the transducers is corrected on the basis of comparison of the time response characteristic stored in the storage unit and the standard time response characteristic of the ultrasound probe, even if the time response characteristics are uneven among the transducers, image quality can be enhanced.

According to the seventeenth aspect of the invention, correction of the echo reception signals matching the time response characteristics of the transducers stored in the storage unit disposed on the ultrasound probe can be performed.

According to the eighteenth aspect of the invention, since correction of the echo reception signals matching the time response characteristics of the transducers measured in advance is made possible, image quality can be enhanced.

Further objects and advantages of the present invention will be apparent from the following description of the preferred embodiments of the invention as illustrated in the accompanying drawings.

### BRIEF DESCRIPTION OF THE DRAWINGS

FIG. 1 is a block diagram showing the configuration of the ultrasound diagnostic apparatus in the first mode for implementing the invention.

FIG. 2 is a diagram showing the electrical resistance R1 of the thermistor at the temperature T1 and the other electrical resistance R2 of the thermistor at the temperature T2 stored in the storage unit.

FIG. 3 is a block diagram showing the configuration of the transceiver unit.

FIG. 4 is a block diagram showing the configuration of the received wave beam former.

FIG. 5 is a block diagram showing a configuration for temperature calculation in the central processing unit.

FIG. 6 is a diagram showing the temperature calculation function f.

FIG. 7 is a block diagram showing the configuration of the ultrasound diagnostic apparatus in the second mode for implementing the invention.

FIG. 8 is a diagram showing the waveform of the frequency characteristic of a certain transducer.

FIG. 9 is a diagram showing a table of the frequency characteristics of the transducers stored in the storage unit.

FIG. 10 is a diagram showing the waveform of the frequency characteristic set with respect to the ultrasound probe.

FIG. 11 is a block diagram showing the configuration of the received wave beam former in the second mode for implementation.

FIG. 12 is a diagram illustrating comparison of the frequency characteristics stored in the storage unit and the set frequency characteristic stored in the main memory.

FIG. 13 is a diagram showing the waveform of the time response characteristic of a certain transducer.

FIG. 14 is a diagram showing a table of the time response characteristics of the transducers stored in the storage unit.

FIG. 15 is a diagram showing the waveform of the standard time response characteristic of the ultrasound probe.

### DETAILED DESCRIPTION OF THE INVENTION

Modes for carrying out the present invention will be described in further detail below with reference to drawings.

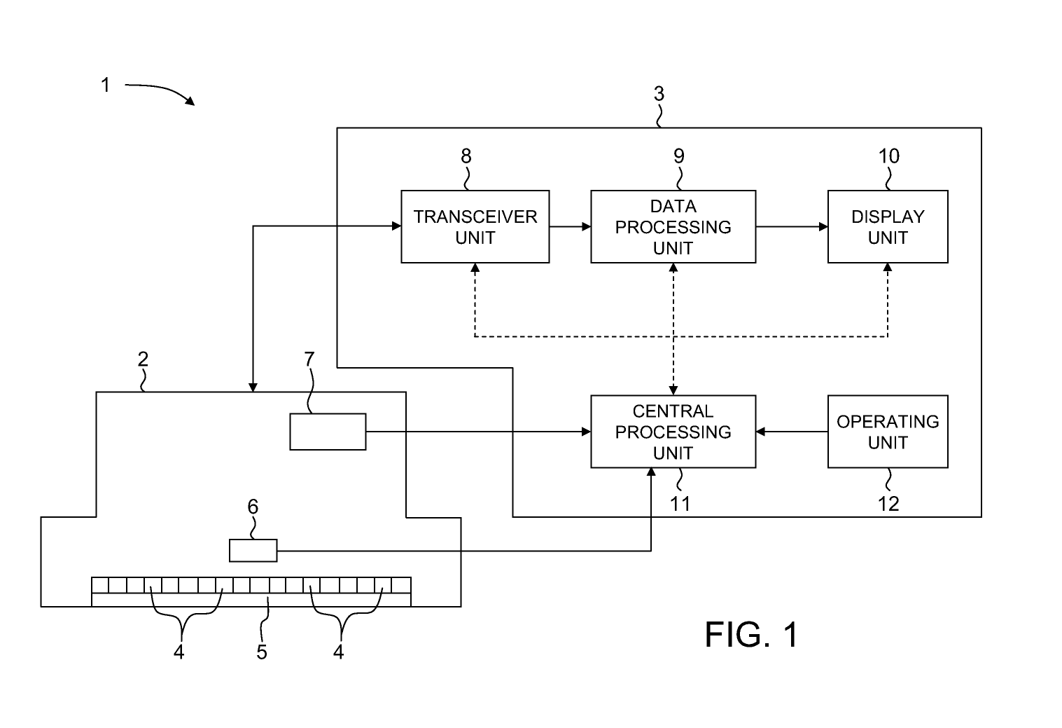

First, a first mode for implementing the invention will be described. FIG. 1 is a block diagram showing the configuration of an ultrasound diagnostic apparatus in the first mode for implementing the invention.

The ultrasound diagnostic apparatus 1 shown in FIG. 1 is so configured as to have an ultrasound probe 2 and an apparatus body 3 to which this ultrasound probe 2 is connected.

The ultrasound probe 2 is provided with a plurality of transducers 4 which transmit and receive ultrasound waves. The number of these transducers may be 64 for instance. The transducers 4 are composed of a piezoelectric material, such as PZT (lead zirconium titanate) or the like. The ultrasound probe 2 is provided with a lens 5 that controls the directivity of transmitted/received ultrasound waves in the transducers 4 and a temperature sensor 6 for detecting the surface temperature of the ultrasound probe 2.

The temperature sensor 6 is disposed within the case (reference sign is omitted) of the ultrasound probe 2 and in the vicinity of the transducers 4. The temperature sensor 6 is connected to the central processing unit 11 (to be described afterwards) of the apparatus body 3. In this mode for implementation, the temperature sensor 6 is a thermistor, and the physical quantity that varies according to the temperature obtained by the temperature sensor 6 is electrical resistance.

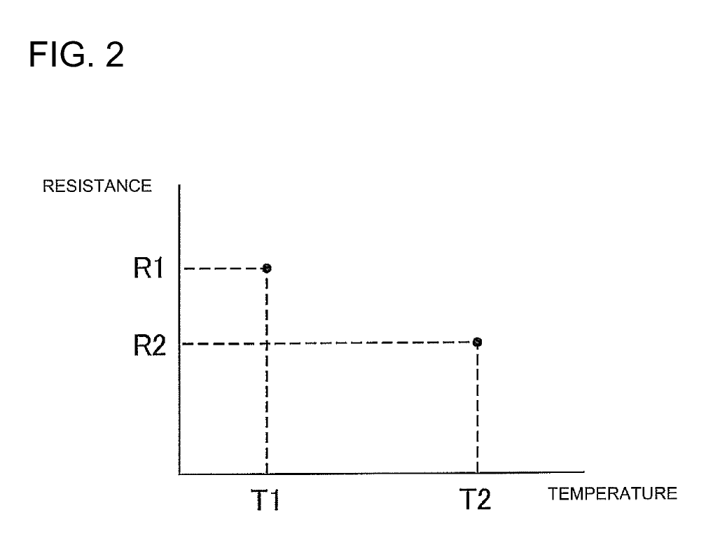

A storage unit 7 is disposed in the ultrasound probe 2. As this storage unit 7, a nonvolatile memory, such as an EEPROM or the like, is used. And in this storage unit 7, temperature characteristics of the temperature sensor 6 measured in advance are stored to perform temperature calculation matching the temperature characteristics of the temperature sensor 6. In this example, as the temperature characteristics of the temperature sensor 6 measured in advance, physical quantities at specific temperatures measured in advance are supposed to be stored. More specifically, an electrical resistance R1 of the thermistor at a temperature T1 and another electrical resistance R2 of the thermistor at a temperature T2, both measured in advance, are stored as the physical quantities at the specific temperature as shown in FIG. 2.

The apparatus body 3 has a transceiver unit 8, a data processing unit 9, a display unit 10, the central processing unit 11 and an operating unit 12. Each constituent unit will be described below.

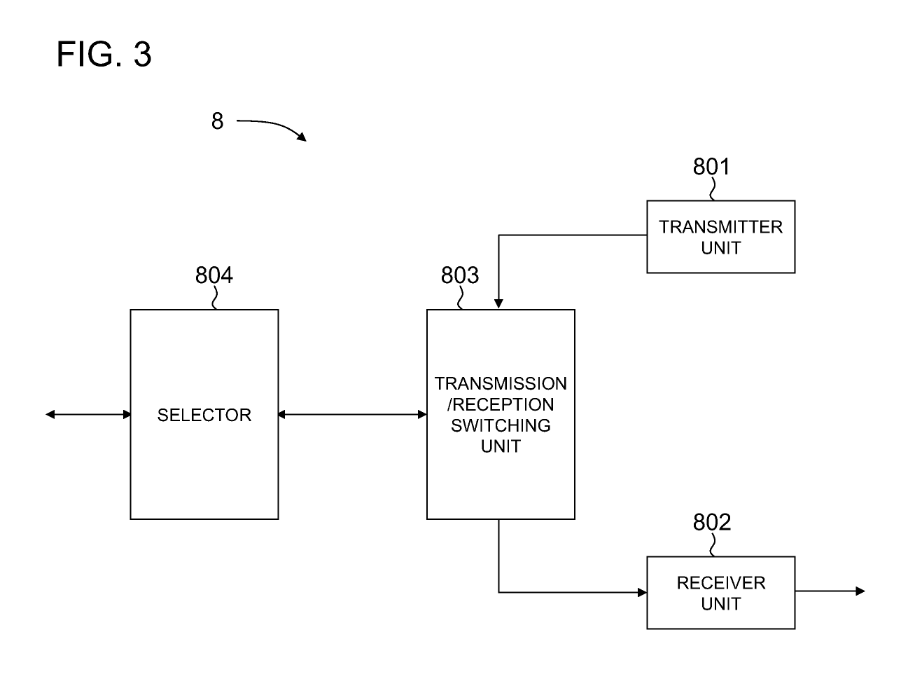

First, the transceiver unit 8 will be described with reference to FIG. 3. FIG. 3 is a block diagram showing the configuration of the transceiver unit 8. The transceiver unit 8 comprises a transmitter unit 801, a receiver unit 802, a transmission/reception switching unit 803 and a selector 804. The transmitter unit 801 is so configured as to output to the transmission/reception switching unit 803 a plurality of driving signals for driving the plurality of transducers 4. The transmission/reception switching unit 803, when the driving signals are inputted from the transmitter unit 801, is so configured as to output them to the selector 804. And the selector 804 is so configured as to select a plurality of transducers 4 to form a transmission aperture out of the transducers 4, and to supply driving signals to them.

Also, the selector 804 is so configured as to select a plurality of transducers 4 to form a reception aperture out of the transducers 4, and to output to the transmission/reception switching unit 803 a plurality of echo signals received by those transducers 4.

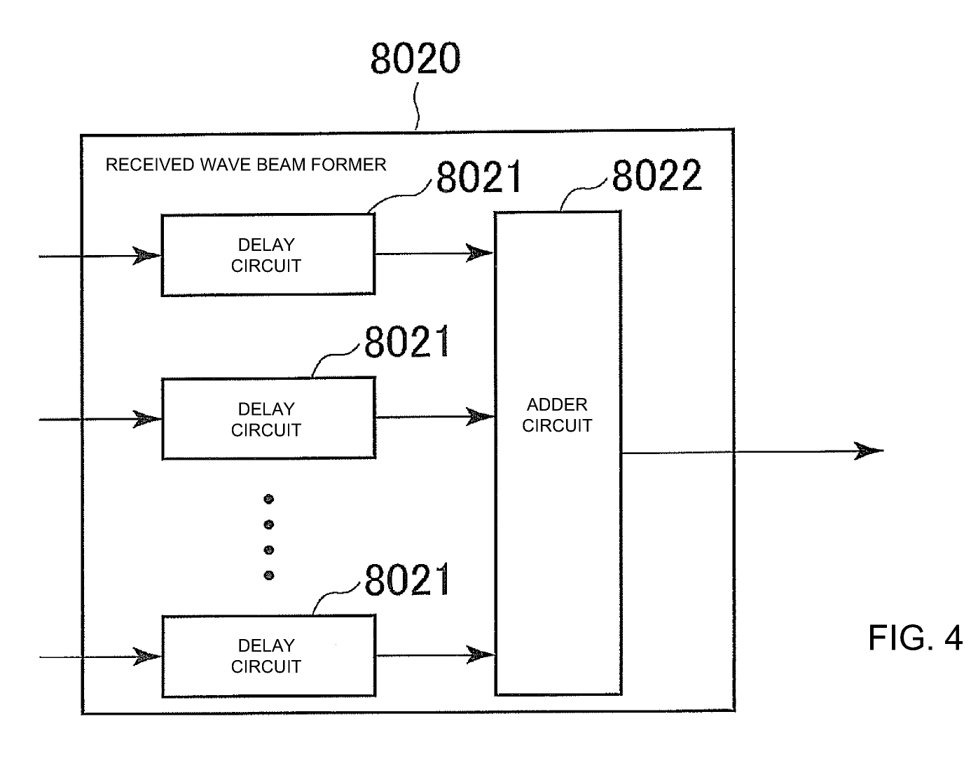

The transmission/reception switching unit 803 is so configured as to input, when the plurality of echo signals are inputted from the selector 804, those signals to the receiver unit 802. The receiver unit 802, having a received wave beam former 8020 as shown in FIG. 4, is so configured as to adjust phases by assigning a time difference to each of the echo signals in a delay circuit 8021 in this received wave beam former 8020, and then to form an echo received signal for each of reception sound rays by adding them in an adder circuit 8022 in the received wave beam former 8020.

The receiver unit 802, connected to the data processing unit 9, is so configured as to cause the echo received signal for each reception sound ray to be inputted to the data processing unit 9. The data processing unit 9 subjects the echo received signal for each reception sound ray to prescribed processing to form data for displaying on the display unit 10 as an image.

These transceiver unit 8, data processing unit 9 and display unit 10 are connected to the central processing unit 11. The central processing unit 11 is so configured as to output control signals to these units to control their operations.

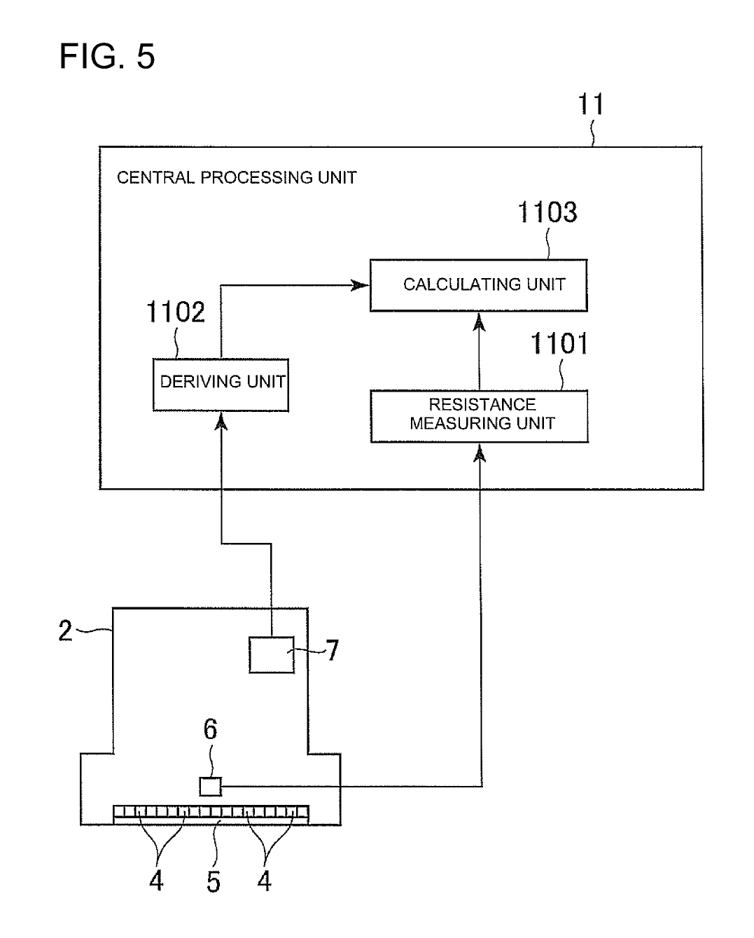

Also, the central processing unit 11, besides having such a function as a control device, is so configured as to calculate the temperature in response to an output signal from the temperature sensor 6 and to so control, on the basis of the calculated temperature, driving power supplied from the transmitter unit 801 to the transducers 4 as to keep the surface temperature of the ultrasound probe 2 at or below a prescribed temperature. FIG. 5 is a block diagram showing a configuration for temperature calculation in the central processing unit 11. The central processing unit 11 is provided with a resistance measuring unit 1101 which measures electrical resistances as the physical quantities of the temperature sensor 6 which varies with the temperature, a deriving unit 1102 which derives a temperature calculation function for calculating the temperature, and a calculating unit 1103 which calculates the temperature by substituting the electrical resistances obtained by the resistance measuring unit 1101 into the temperature calculation function obtained by this deriving unit 1102. In this mode for implementation, the temperature calculation function is a primary function representing the relationship between electrical resistance and temperature. Also the central processing unit 11 has a function as a control unit for controlling driving power from the transmitter unit 801 to the transducers 4 on the basis of the temperature calculated by the calculating unit 1103. Driving power control for the resistance measuring unit 1101, the deriving unit 1102, the calculating unit 1103, the temperature calculation function and the transducers 4 will be described in detail afterwards.

The operating unit 12, connected to the central processing unit 11, is so configured as to be operated by the operator to input desired instructions and information to the central processing unit 11. The operating unit 12 is configured of an operation panel provided with, for instance, a keyboard and other operating tools.

Now, the operation of the ultrasound diagnostic apparatus 1 will be described. In the ultrasound diagnostic apparatus 1 when transmitting or receiving an ultrasound wave, the central processing unit 11 calculates the temperature in response to an output signal from the temperature sensor 6, and controls on the basis of the calculated value the driving power to be supplied to the transducers 4. A more specific description will follow.

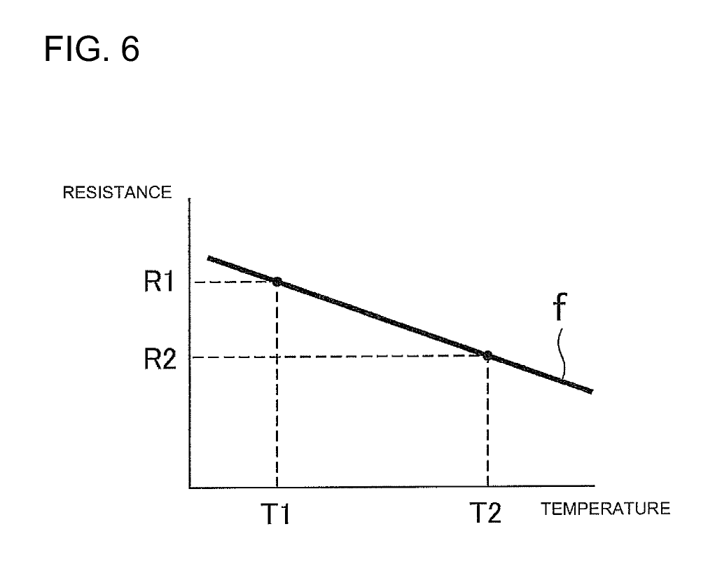

First, the calculation of the temperature will be described. The resistance measuring unit 1101 of the central processing unit 11 measures the electrical resistance on the basis of the input from the temperature sensor 6, and outputs the obtained resistance value to the calculating unit 1103. The deriving unit 1102, after extracting the electrical resistance R1 at the temperature T1 and the electrical resistance R2 at the temperature T2, stored in the storage unit 7 disposed in the ultrasound probe 2, derives from these T1, T2, R1 and R2, a temperature calculation function f shown in FIG. 6, and outputs this temperature calculation function f to the temperature calculating unit 1103.

The temperature calculating unit 1103, having received the resistance value from the resistance measuring unit 1101 and the temperature calculation function f from the deriving unit 1102, substitutes the resistance value into the temperature calculation function f to calculate the temperature. As the temperature calculation function f here is derived on the basis of the temperature characteristics stored in the storage unit 7, namely the electrical resistance R1 at the temperature T1 and the electrical resistance R2 at the temperature T2, the temperature calculation by the calculating unit 1103 is a temperature calculation based on the temperature characteristics stored in the storage unit 7.

The central processing unit 11 so controls the driving power to be supplied from the transmitter unit 801 as to keep the surface temperature of the ultrasound probe 2 at or below a prescribed temperature on the basis of the calculated value.

As the ultrasound diagnostic apparatus 1 in the first mode for implementation so far described derives the temperature calculation function f here on the basis of temperature characteristics measured in advance regarding the temperature sensor 6, namely the electrical resistance R1 at the temperature T1 and the electrical resistance R2 at the temperature T2, an accurate temperature calculation function f regarding the temperature sensor 6 can be derived. Therefore, as the temperature calculation matching the temperature characteristics of the temperature sensor 6 can be accomplished by calculating the temperature by the use of such a temperature calculation function f, the surface temperature of the ultrasound probe 2 can be accurately determined.

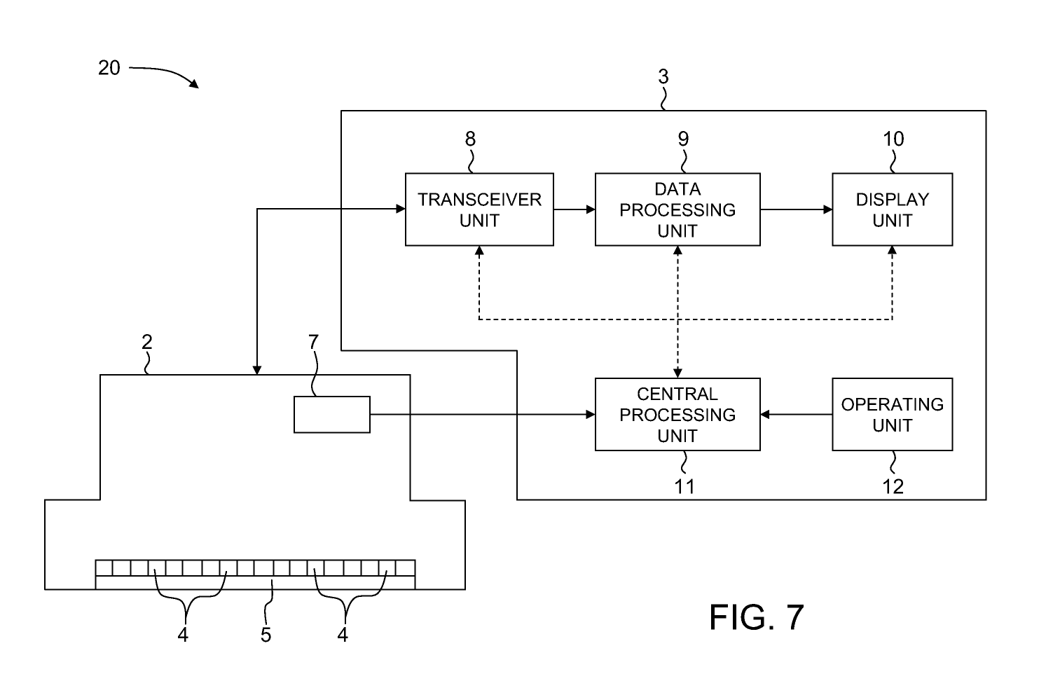

Next, a second mode for implementing the present invention will be described. FIG. 7 is a block diagram showing the configuration of an ultrasound diagnostic apparatus in the second mode for implementing the invention.

An ultrasound diagnostic apparatus 20 in the second mode for implementation, though the same as the ultrasound diagnostic apparatus 1 in the first mode for implementation in basic configuration, differs from the ultrasound diagnostic apparatus 1 in that the ultrasound probe 2 is not provided with a temperature sensor.

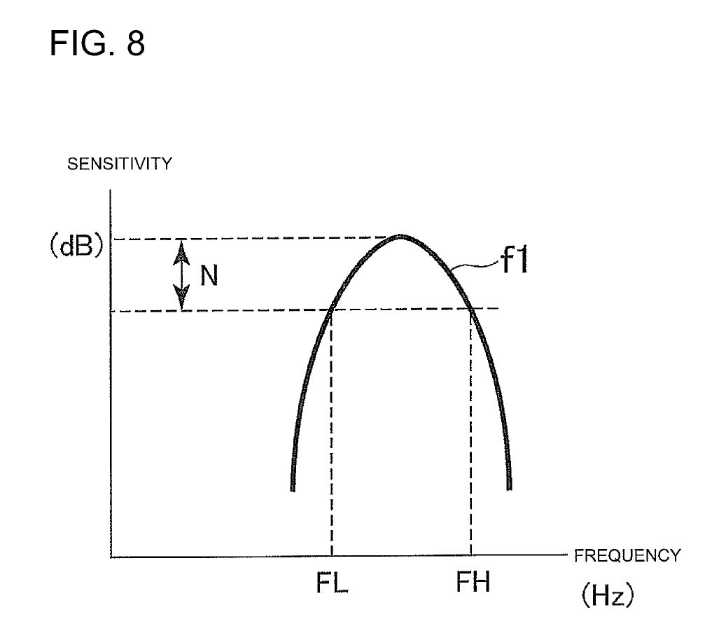

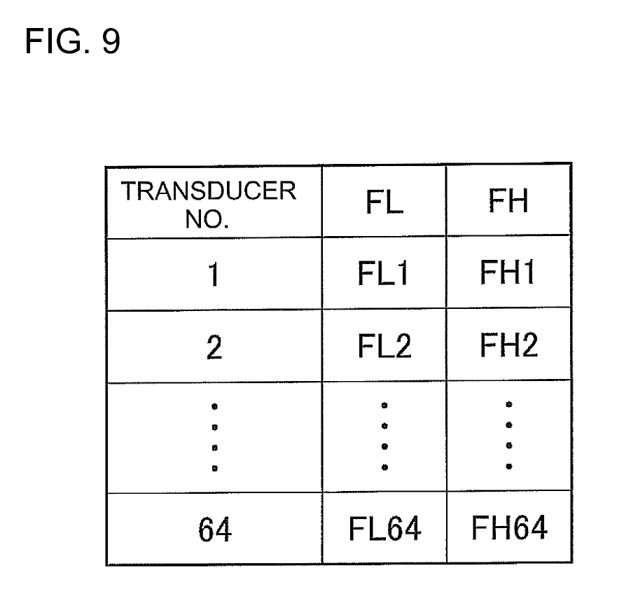

In this mode for implementation, the frequency characteristics of the transducers 4 measured in advance are stored in the storage unit 7 to so perform correction of echo received signals as to match the frequency characteristics of the transducers 4. A more specific description will be given with reference to FIG. 8. FIG. 8 is a diagram showing the waveform of the frequency characteristic of a certain transducer 4. In this mode for implementation, the frequency characteristic of each transducer is measured, and, in the waveform f1 showing the obtained frequency characteristic, a frequency FL and a frequency FH corresponding to a point of attenuation by a prescribed quantity N dB (e.g. 6 dB) from the maximum sensitivity are stored into the storage unit 7. The frequency characteristics of the transducers 4 are stored in the storage unit 7 in the form of a table as shown in FIG. 9 for instance. More specifically, a frequency FL1, and a frequency FH1, a frequency FL2 and a frequency FH2, . . . , a frequency FL64 and a frequency FH64, are stored respectively for transducers numbered 1 through 64.

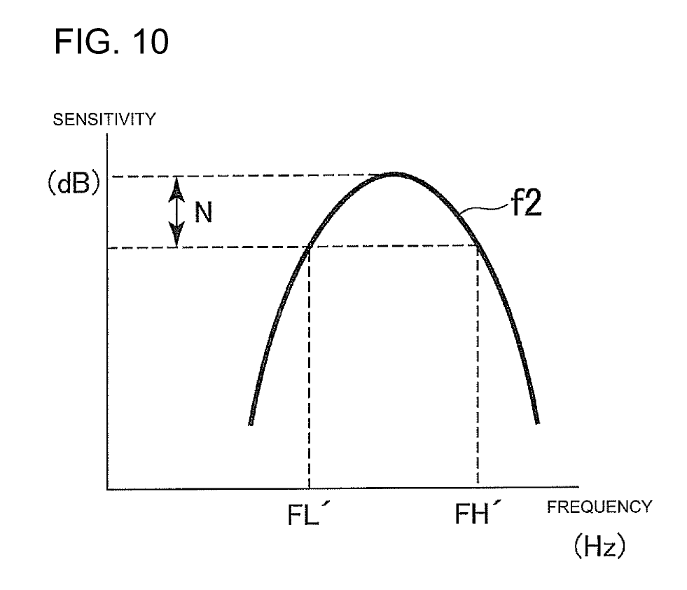

Also, the set frequency characteristic of the ultrasound probe 2 is stored in the main memory (not shown) of the apparatus body 3. This frequency characteristic is set according to the purpose of use of the ultrasound probe 2 or the like. A more specific description will be given with reference to FIG. 10. FIG. 10 is a diagram showing the waveform of the frequency characteristic set with respect to the ultrasound probe 2. In the main memory of the apparatus body 3, a frequency FL′ and a frequency FH′, corresponding to a point of attenuation by a prescribed quantity N dB (e.g. 6 dB) from the maximum sensitivity in the waveform f2 shown in FIG. 10 are stored as the set frequency characteristics of the ultrasound probe 2.

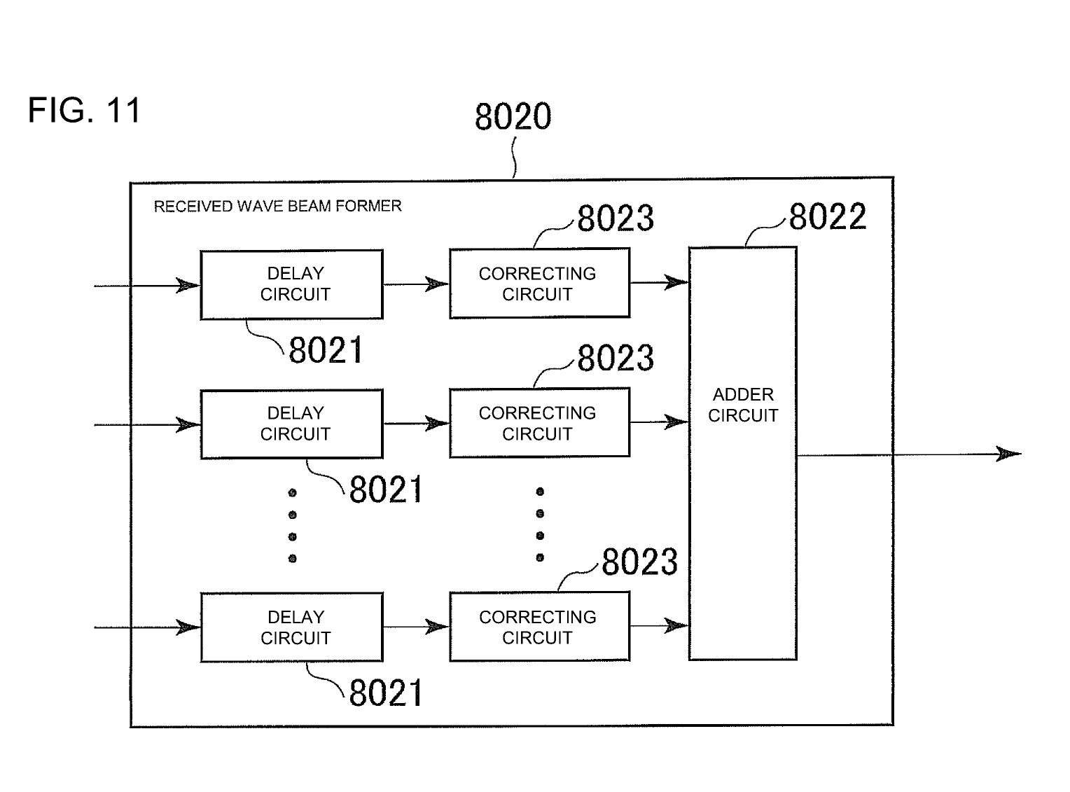

FIG. 11 is a block diagram showing the configuration of a received wave beam former 8020 in the second mode for implementation. The received wave beam former 8020 in this mode for implementation has correcting circuits 8023 which correct echo received signals for each of the transducers 4 on the basis of the frequency characteristics of the transducers 4 stored in the storage unit 7. These correcting circuits 8023 are disposed between the delay circuits 8021 and the adder circuit 8022. The correcting circuits 8023 are so configured as to receive correction signals from the central processing unit 11 and to correct echo reception signals. The correction of echo reception signals by the correcting circuits 8023 will be described in detail afterwards.

The operation of the ultrasound diagnostic apparatus 20 in this mode for implementation will be described. In the following description, different aspects of operation from the ultrasound diagnostic apparatus 1 in the first mode for description will be described.

When generating an image on the basis of echo reception signals, the ultrasound diagnostic apparatus 20 corrects the echo reception signal of each transducer 4 on the basis of the frequency characteristic of each of the transducers 4 stored in the storage unit. Detailed description will follow.

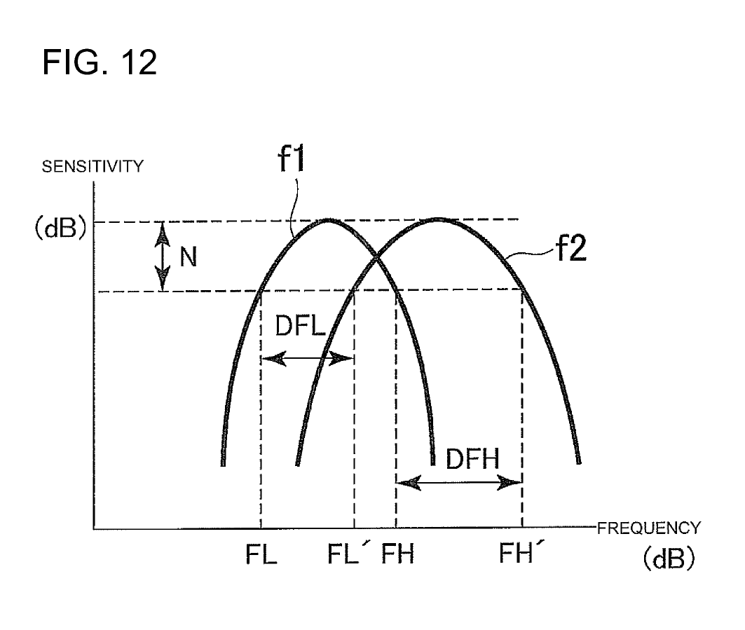

The central processing unit 11 compares the frequency characteristic stored in the storage unit 7 for each transducer 4 and the set frequency characteristic stored in the main memory (not shown) of the apparatus body 3, and outputs correction signals generated on the basis of this comparison to the correcting circuits 8023. A more specific description will be given with reference to FIG. 12. The central processing unit 11 performs calculation of a difference DFL between the frequency FL stored in the storage unit 7 and the frequency FL′ stored in the main memory and a difference DFH between the frequency FH and the frequency FH′ as comparison of the frequency characteristics stored in the storage unit 7 and the set frequency characteristic stored in the main memory. The central processing unit 11 performs calculation of the DFL and the DFH for each of the transducers 4. And the central processing unit 11 calculates the quantity of shift in the direction of the frequency axis with respect to echo reception signals in the transducers 4 by a known arithmetic method on the basis of the DFL and the DFH calculated in this way, generates a correction signal for shifting each of the transducers 4 by this quantity of shift in the direction of the frequency axis, and outputs this correction signal to the corresponding correcting circuit 8023.

The correcting circuit 8023 corrects echo reception signals on the basis of the correction signals generated on the basis of comparison of the frequency characteristics stored in the storage unit 7 and the set frequency characteristic stored in the main memory, namely in accordance with the correction signals on the basis of the DFL and the DFH.

The ultrasound diagnostic apparatus 20 in the second mode for implementation so far described, since the frequency characteristics of the transducers 4 are stored and the echo reception signal of each of the transducers 4 is corrected on the basis of comparison of these frequency characteristics and the set frequency characteristic of the ultrasound probe 2, even if the frequency characteristics are uneven among the transducers 4, image quality can be enhanced.

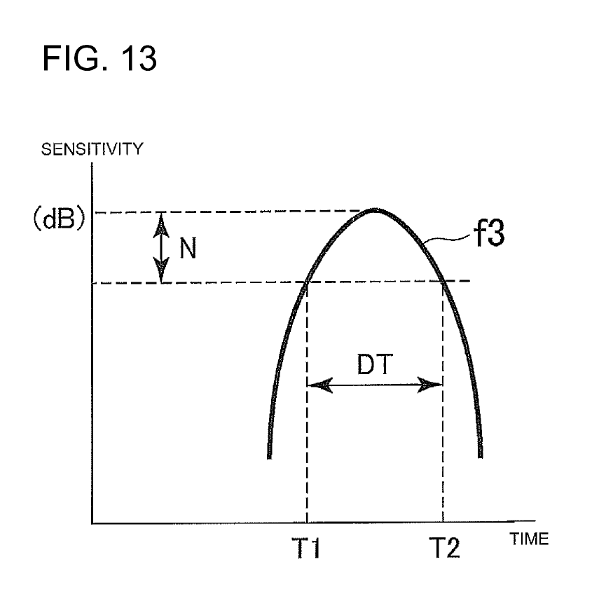

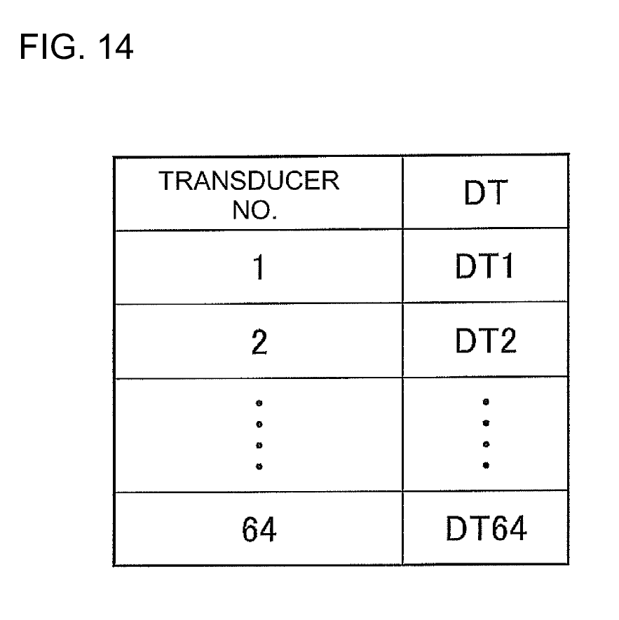

Next, a modified version of the second mode for implementation will be described. The time response characteristics of the transducers stored in advance in the storage unit 7 of this modified version of the ultrasound diagnostic apparatus 20 to perform correction of echo reception signals matching the time response characteristics of the transducers 4. A more specific description will be given with reference to FIG. 13. FIG. 13 is a diagram showing the waveform of the time response characteristic of a certain transducer 4. In this FIG. 13, the time on the horizontal axis represents the length of time from the transmission of an ultrasound wave by the transducer 4 until the reception of an echo signal responding to it, and the time response characteristic means the relationship between the time and the sensitivity from the transmission until the reception of the ultrasound wave. In this mode for implementation, the time response characteristic of each of the transducers 4 is measured, and the length of time from the time DT of the part of attenuation by a prescribed quantity N dB (e.g. 6 dB) from the maximum sensitivity in a waveform f3 representing the obtained time response characteristic, namely the length of time from a time T1 of the part in which attenuation by N dB has taken place from the maximum sensitivity until a time T2 by which attenuation by N dB has taken place again via the maximum sensitivity, is stored into the storage unit 7. The time response characteristic of each of the transducers 4 is stored into the storage unit 7 as a table shown in FIG. 14 for instance. More specifically, a time response characteristic DT1, 1, a time response characteristic DT2, . . . , a time response characteristic DT64 are stored for each of the transducers 1 through 64.

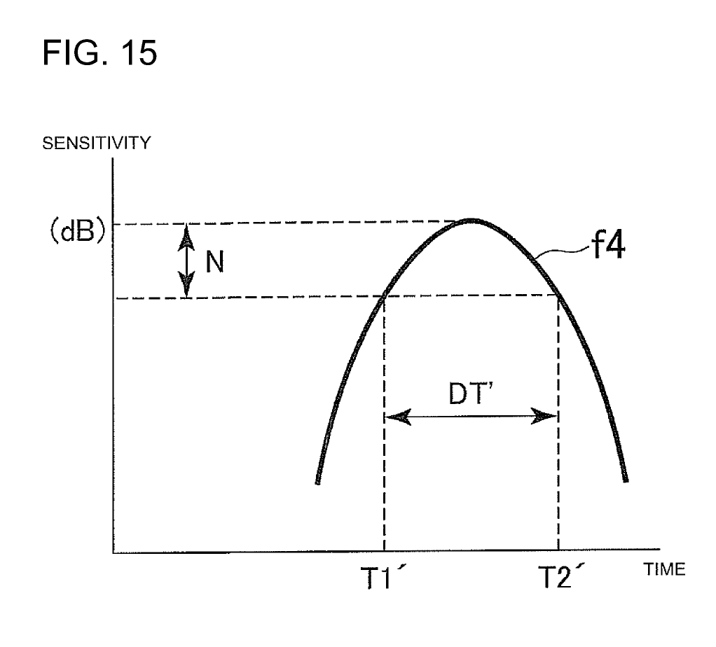

Also, the standard time response characteristic of the ultrasound probe 2 is stored in the main memory (not shown) of the apparatus body 3. The standard time response characteristic here means the general time response characteristic of the transducers 4 in a living body. A more specific description will be given with reference to FIG. 15. FIG. 15 is a diagram showing the waveform of the standard time response characteristic of the ultrasound probe 2. In the main memory of the apparatus body 3, the length of time from the time DT′ of the part of attenuation by a prescribed quantity N dB (e.g. 6 dB) from the maximum sensitivity in a waveform f4 shown in FIG. 15, namely the length of time from a time T1′ of the part in which attenuation by N dB has taken place from the maximum sensitivity until a time T2′ by which attenuation by N dB has taken place again via the maximum sensitivity, is stored.

Now, the ultrasound diagnostic apparatus 20 in the modified version of the second mode for implementation will be described. In the following description, different aspects of operation from the ultrasound diagnostic apparatus 20 described above will be taken up.

In the ultrasound diagnostic apparatus 20, when an image is to be generated on the basis of echo reception signals, the echo reception signal of each of the transducers 4 is corrected on the basis of time response characteristic of each of the transducers 4 stored in the storage unit 7. A detailed description will follow.

The central processing unit 11 compares the time response characteristic stored in the storage unit 7 for each transducer 4 and the standard time response characteristic stored in the main memory (not shown) of the apparatus body 3, and outputs correction signals generated on the basis of this comparison to the correcting circuits 8023. More specifically, the central processing unit 11 performs calculation of a difference between the time DT stored in the storage unit 7 and the time DT′ stored in the main memory as comparison of the time response characteristics stored in the storage unit 7 and the standard time response characteristic stored in the main memory. The central processing unit 11 performs calculation of the difference between the time DT and the time DT′ for each of the transducers 4. And the central processing unit 11 calculates the quantity of shift in the direction of the time axis with respect to echo reception signals in the transducers 4 by a known arithmetic method on the basis of the difference between the time DT and the time DT′ calculated in this way, generates a correction signal for shifting each of the transducers 4 by this quantity of shift in the direction of the time axis, and outputs this correction signal to the corresponding correcting circuit 8023.

The correcting circuits 8023 correct echo reception signals according to correction signals generated on the basis of comparison between the time response characteristics stored in the storage unit 7 and the standard time response characteristic stored in the main memory, namely according to correction signals generated on the basis of the difference between the time DT and the time DT′.

The ultrasound diagnostic apparatus 20 in the modified version of the second mode for implementation described above, since the time response characteristics of the transducers 4 are stored and the echo reception signal of each of the transducers 4 is corrected on the basis of comparison of these time response characteristics and the standard time response characteristic of the ultrasound probe 2, even if the frequency characteristics are uneven among the transducers 4, image quality can be enhanced.

Although the present invention has been described with reference to different modes for implementation thereof, the invention can obviously implemented in various other ways without deviating from the essentials thereof. For instance, the storage unit 7 may as well be disposed in the apparatus body 3 instead of the ultrasound probe 2.

Many widely different embodiments of the invention may be configured without departing from the spirit and the scope of the present invention. It should be understood that the present invention is not limited to the specific embodiments described in the specification, except as defined in the appended claims.

1. An ultrasound diagnostic apparatus comprising:
a temperature sensor disposed on an ultrasound probe, said temperature sensor configured to detect a temperature of a surface of said probe; and
a calculating unit configured to calculate the temperature based on an input from said temperature sensor;
a storage unit configured to store temperature characteristics of said temperature sensor measured in advance, wherein said calculating unit is configured to perform the temperature calculation according to the temperature characteristics of said temperature sensor stored.

2. The ultrasound diagnostic apparatus according to claim 1, wherein said calculating unit is configured to perform the temperature calculation by substituting a physical quantity, which varies according to the temperature at said temperature sensor, into a temperature calculation function representing a relation between a physical quantity and the temperature of said temperature sensor, wherein said storage unit is configured to store the physical quantity at a specific temperature of said temperature sensor measured in advance, said ultrasound diagnostic apparatus further comprises a deriving unit configured to derive the temperature calculation function based on the physical quantity stored in said storage unit.

3. The ultrasound diagnostic apparatus according to claim 2, wherein:
the physical quantity of said temperature sensor which varies according to the temperature is an electrical resistance, electrical resistances at two or more different specific temperatures measured in advance being stored in said storage unit;
said deriving unit is configured to derive the temperature calculation function representing a relationship between electrical resistances and temperatures at said temperature sensor based on of the electrical resistances at the two or more different specific temperatures stored in said storage unit; and
said calculating unit is configured to perform the temperature calculation by substituting electrical resistances at said temperature sensor into the temperature calculation function.

4. The ultrasound diagnostic apparatus according to claim 1, wherein:
said storage unit is provided in said ultrasound probe.

5. The ultrasound diagnostic apparatus according to claim 2, wherein:
said storage unit is provided in said ultrasound probe.

6. The ultrasound diagnostic apparatus according to claim 3, wherein:
said storage unit is provided in said ultrasound probe.

7. An ultrasound probe comprising including:
a temperature sensor configured to detect a temperature of a surface of said probe; and
a storage unit configured to store temperature characteristics of said temperature sensor measured in advance and to perform a temperature calculation by matching the temperature characteristics of said temperature sensor.

8. A temperature calculating method for use in an ultrasound diagnostic apparatus which is provided with a temperature sensor disposed on an ultrasound probe, said method comprising:
detecting a temperature of a surface of the probe using the temperature sensor;
calculating the temperature based on an input from the temperature sensor; and
temperature calculation matching temperature characteristics of the temperature sensor measured in advance.

9. The temperature calculating method for use in an ultrasound diagnostic apparatus according to claim 8, wherein:
calculating the temperature comprises substituting a physical quantity, which varies according to the temperature at the temperature sensor, into a temperature calculation function representing a relation between a physical quantity and the temperature of the temperature sensor, said method further comprising deriving the temperature calculation function using a physical quantity at a specific temperature of the temperature calculation function measured in advance.

10. The temperature calculating method for use in an ultrasound diagnostic apparatus according to claim 9, wherein:
driving the temperature calculation function comprises using electrical resistances at two or more different specific temperatures being measured in advance; and
calculating the temperature comprises substituting an electrical resistance at the temperature sensor into the temperature calculation function.

11. An ultrasound diagnostic apparatus comprising:
an ultrasound probe comprising a plurality of transducers configured to transmit and receive ultrasound waves;
a receiver unit configured to receive as echo reception signals a reflected wave received by said plurality of transducers;
a storage unit configured to store a frequency characteristic of each of said plurality of transducers measured in advance; and
a correcting unit configured to correct the echo reception signal of each of said plurality of transducers based on the frequency characteristic of each of said plurality of transducers stored in said storage unit.

12. The ultrasound diagnostic apparatus according to claim 11, wherein:
the correction of echo reception signals by said correcting unit is accomplished based on a comparison of the frequency characteristic stored in said storage unit and a set frequency characteristic of said ultrasound probe.

13. The ultrasound diagnostic apparatus according to claim 11, wherein:
said storage unit is disposed in said ultrasound probe.

14. The ultrasound diagnostic apparatus according to claim 12, wherein:
said storage unit is disposed in said ultrasound probe.

15. An ultrasound probe comprising:
a plurality of transducers configured to transmit and receive ultrasound waves; and
a storage unit configured to store a frequency characteristic of each of said plurality of transducers measured in advance and to perform correction matching the frequency characteristics of said plurality of transducers regarding the echo reception signals received by said plurality of transducers.

16. An ultrasound diagnostic apparatus comprising:
an ultrasound probe comprising a plurality of transducers configured to transmit and receive ultrasound waves;
a receiver unit configured to receive as echo reception signals a reflected wave received by said, plurality of transducers;
a storage unit configured to store a time response characteristic of each of said plurality of transducers measured in advance; and
a correcting unit configured to correct the echo reception signal of each of said plurality of transducers based on the time response characteristic of each of said transducers stored in said storage unit.

17. The ultrasound diagnostic apparatus according to claim 16, wherein:
the correction of echo reception signals by said correcting unit is accomplished based on a comparison of the time response characteristic stored in said storage unit and a standard time response characteristic of said ultrasound probe.

18. The ultrasound diagnostic apparatus according to claim 16, wherein:
said storage unit is disposed in said ultrasound probe.

19. The ultrasound diagnostic apparatus according to claim 17, wherein:
said storage unit is disposed in said ultrasound probe.

20. An ultrasound probe comprising:
a plurality of transducers configured to transmit and receive ultrasound waves; and
a storage unit configured to store a time response characteristic of each of said plurality of transducers measured in advance and to perform correction matching the time response characteristics of said plurality of transducers regarding the echo reception signals received by said plurality of transducers.
